(19) United States
(12) Patent Application Publication
(10) Pub. No.: US 2024/0041622 A1
(43) Pub. Date: Feb. 8, 2024

(54) IMPLANT AND ASSEMBLY HAVING A RADIATION SOURCE AND AN IMPLANT

(71) Applicant: RHEINISCH-WESTFÄLISCHE TECHNISCHE HOCHSCHULE (RWTH) AACHEN, Aachen (DE)

(72) Inventors: Benedict BAUER, Aachen (DE); Thomas Gerhard GRIES, Aachen (DE); Jeanette ORTEGA, Aachen (DE); loana SLABU, Aachen (DE); Thomas SCHMITZ-RODE, Aachen (DE); Benedikt MUES, Aachen (DE)

(73) Assignee: RHEINISCH-WESTFÄLISCHE TECHNISCHE HOCHSCHULE (RWTH) AACHEN, Aachen (DE)

(21) Appl. No.: 18/256,757

(22) PCT Filed: Dec. 21, 2021
(86) PCT No.: PCT/EP2021/087083

§ 371 Date: Jun. 9, 2023

(30) Foreign Application Priority Data
Dec. 22, 2020 (DE) 10 2020 134 541.0

(51) Int. Cl. A61F 2/90; A61N 1/40

(52) U.S. Cl. CPC A61F 2/90; A61N 1/403

(57) ABSTRACT

The present invention relates to an implant for implanting in a body, in particular in a hollow organ or a vessel of a body, the implant being composed of a filament which comprises at least one polymeric matrix material in which a magnetically heatable filler is arranged, the filament having a cross section with a core-sheath structure characterized in that the core forms a polymeric reinforcing structure, and in that the sheath comprises the polymeric matrix material in which the magnetically heatable filler is disposed, the loading of the filler being greater in the sheath than in the core.

The present invention relates to an implant, such as in particular a stent. In particular, the present invention relates to an implant applicable in magnetically induced hyperthermia. The present invention further relates to an arrangement comprising such an implant and a radiation source for emitting electromagnetic radiation.

Therapeutic hyperthermia is known in itself and can achieve significant benefits in cancer treatment, for example.

For example, it is known from I. Slabu, MPI visualization and inductive heating of hybrid implant fibers, International Journal on Magnetic Particle Imaging, Vol 6, No 2, Suppl 1, Article ID 2009024, and from I. Slabu, Assessing hyperthermia performance of hybrid textile filaments: the impact of different heating agents, Journal of Magnetism and Magnetic Materials, 519 (2021) 167486, that polypropylene fibers, which have magnetic nanoparticles added, can generate effective therapeutic heat. In particular, such fibers can be used for inductively heatable stents in cancer therapy. Furthermore, an application in so-called Magnetic Particle Imaging (MPI) is described.

EP 1 489 985 B1 describes the use of a material in a vascular treatment device having a magnetic susceptibility that is heat sensitive. The vascular treatment device can then be remotely and non-invasively heated, using an applied magnetic field, to a preselected temperature at which the vascular treatment device becomes substantially non-magnetically susceptible. In this regard, the material may be provided, for example, as a coating on a stent, and the core of the stent may be, for example, a metal. In the case where the stent is formed from a polymer, the material may be embedded in the polymer or the pure material may be coated onto the polymer. A two-layer polymer structure is not described in this document.

US 2003/0004563 A1 describes a stent for implantation into a body. With regard to the structure of the stent, this document describes that it can be composed of a polymer which is mixed with an additive. The additive may comprise particles of a metal, for example, and/or a radiopaque material, for example in the nanometer range. In particular, paramagnetic or ferromagnetic materials may be used to be visible in an MRI process. Alternatively, it may be envisaged that a double layer structure is used such that there is an inner polymeric core to which a layer of the pure MR material is applied. The polymeric backbone can be produced, for example, by melt spinning. This document also does not describe a two-layer polymer structure.

US 2010/0087731 A1 describes a tubular stent formed from a plurality of filaments, the filaments being composed of a solid, bioabsorbable polymer material with drug particles dispersed therein that are visible by magnetic resonance imaging (MRI). The drug particles are superparamagnetic iron oxide (SPIO) particles. The SPIO particles enhance the visibility of the polymer stent under MRI and also allow accurate monitoring of stent degradation. As the stent degrades, the SPIO particles are released and either flow downstream or are embedded by nearby macrophages. The amount of SPIO particles within the remaining stent body is reduced, resulting in a different MRI signal. By quantifying the signal change, the amount of remaining biodegradable stent in situ can be derived and the stent degradation rate can be accurately calculated. A two-layer polymer design is also not described in this document.

US 2016/0024699 A1 describes a melt-spun fiber in which an additive is provided that is detectable magnetically or via X-rays and has a size of less than 31 micrometers. The additive may be, for example, a metal or a material that is opaque to X-rays. The additive may be present in the core and/or the sheath of a polymer matrix. Such fibers are used in a wide variety of products, in particular to detect contamination of manufactured products with these fibers. However, this document does not describe an implant, nor does it describe the possibility of forming a tubular structure.

DE 10 2018 005 070 A1 relates to a method of manufacturing a stent graft. The method comprises providing a graft of a first polymer-based material and applying a stent structure comprising a plurality of struts of a second polymer-based material to the graft by means of an additive manufacturing process. The invention further relates to a stent graft fabricated by the method. However, in such a stent graft, the overall tubular structure is provided with struts, and no multilayer structure is described at the fiber level.

The document “Investigation of nanoferrite-based inductive fibers for applications in hyperthermic ablation therapy” which is a publication also by inventors of the present application, describes the application of inductively heatable nanoferrites in stents, generated from nanocomposite fibers, in cancer therapy and more precisely in the fight against tumors. This document does not describe a core-sheath structure.

Such solutions known from the prior art may still have potential for improvement, especially with regard to improved applicability.

It is therefore the object of the present invention to create a measure by which at least one disadvantage of the prior art is at least partially overcome. In particular, it is an object of the present invention to provide a measure by means of which the applicability of an implant, in particular a stent, can be improved.

According to the invention, the object is solved by an implant with the features of claim 1. According to the invention, the object is further solved by an arrangement with the features of claim 13. Preferred embodiments of the invention are disclosed in the dependent claims, in the description, and in the figures, wherein further features described or shown in the dependent claims or in the description or the figures may individually or in any combination constitute an object of the invention, unless the opposite clearly results from the context.

The present invention relates to an implant for implanting in a body, in particular in a hollow organ or a vessel of a body, the implant being composed of a filament which comprises at least one polymeric matrix material in which a magnetically heatable filler is arranged, the filament having a cross section with a core-sheath structure wherein the core forms a polymeric reinforcing structure, and wherein the sheath comprises the polymeric matrix material in which the magnetically heatable filler is disposed, the loading of the filler being greater in the sheath than in the core.

In particular, such an arrangement can offer significant advantages over prior art solutions, such as for use in hyperthermia or hyperthermal therapy, or in imaging using magnetic resonance imaging (MRI) or magnetic particle imaging (MPI).

The implant described herein serves in particular for implantation into a body, in particular of a living being, such as in a human body, wherein implantation into a vessel or hollow organ of the body, such as for example into a blood vessel, the trachea, bile ducts, and ureters and urethra, is particularly preferred, as will be described in greater detail later. For this purpose, the implant may in particular have a flexibility to be introduced into the body, such as into the vessel or hollow organ, for example in a compressed and/or deformed state, and to accomplish its desired application form at the desired position.

The implant is formed from a filament, which may also be referred to as a fiber. To manufacture the implant, the filament can be processed in a manner known per se using fiber processing methods familiar to the skilled person. Examples of such fiber processing methods or textile-technical further processing processes include, for example, the interlacing or intertwining of the filament, as occurs in weaving, warp knitting, knitting, lace manufacture, braiding and the manufacture of tufted products. Furthermore, the implant may be a nonwoven, although it may be preferred that the filament is not a nonwoven.

Beforehand, the filament can be created by a spinning process. For this purpose, a coextrusion can be used to create the core-sheath structure.

In particular, but not limited to, a coextrusion process makes it possible to create a core-sheath structure at the fiber level. It is preferred that the core is thread-like and the sheath has a hose-like structure and at least partially envelops the core. In other words, the filament from which the implant is formed, in particular by a fiber processing method, is formed as a multilayer structure at the fiber level. The core is filamentary and thus formed from solid material. The sheath is tubular and formed around the core. This is possible, for example, by using two coaxial dies in an extrusion process, forming the core on the inside and the sheath as a tubular structure radially around the core.

For the purposes of the present invention, a hose-like structure is to be understood in particular as meaning that the sheath has a tubular structure, so that the sheath covers the core outwardly, in particular completely or over the entire surface, in that the sheath runs around the core. The interior of the tubular form is then in particular completely filled by the core. In particular, the core as well as the sheath can preferably form a closed layer, the layers preferably being free of pores. However, pores in the core or in the sheath are generally intended to be encompassed by the present invention.

A coextrusion process for manufacturing the filament can achieve a particularly high strength because the materials, especially polymer materials, are oriented. This is an advantage over an additive manufacturing process, for example, in which the materials are usually unoriented.

The filament has at least one polymeric matrix material in which a magnetically heatable filler is arranged. In particular, the magnetically heatable filler can be homogeneously finely distributed in the matrix material, whereby in the implant described here it is intended that the filler is present only in a predefined region along the cross-section of the filament. The more homogeneous the distribution of the filler, the more homogeneous and defined the hyperthermal therapy can be carried out.

With regard to the magnetically heatable filler, in the sense of the present invention this should in particular be a filler which heats up when triggered by a magnetic field or an electromagnetic field. The heating takes place in particular in a defined and reproducible manner, so that when a magnetic field with known parameters is applied, such an effect is achieved that the filler or in particular the filament can be heated to a defined temperature value. With regard to an application for hyperthermia, as described in detail later, it is advantageous if the filler can be heated in such a way that the filament can obtain a temperature in a range from 40° C. to 100° C., preferably in a range from 41° C. to 44° C. In this context, it can be particularly advantageous that, according to the invention, even very narrow temperature ranges, such as in a range from 41° C. to 44° C., or also other temperature ranges lying in particular in the aforementioned ranges can be set in a very defined manner. This is possible in particular by the selection and loading of the fillers and the parameters of their magnetic excitation.

Among the above temperature ranges, high temperatures in particular could allow thermoablative procedures to be performed, analogous to high-intensity focused ultrasound (HIFU), radiofrequency-induced thermotherapy (HITT), or laser-induced interstitial thermotherapy (LITT). Thermoablative procedures aim at killing (coagulation) of the target tissue with temperatures of about 80-100° C. or in low temperature ranges, such as in a range of 41° C. to 44° C. with, in particular, apoptotic cell damage, while avoiding undesired necrosis. The implant according to the invention can in principle be used in thermoablative procedures, in particular in the lower temperature range, to generate in particular therapeutically effective heat or for imaging.

With regard to the arrangement of the filler, the implant or filament described herein is characterized in that the filament as described above has a cross-section with a core-sheath structure, the core forming a polymeric reinforcing structure, and the sheath comprising the polymeric matrix material in which the magnetically heatable filler is arranged, the loading of the filler in the sheath being greater than in the core. The core may further also comprise the matrix material provided in the sheath, or be formed from a different polymer.

Thus, the magnetically heatable filler is predominantly present in the outer region, whereas the core contains less of the magnetically heatable filler or, in particular, is free of it. As a result, a polymeric reinforcing structure is formed in the core. For the purposes of the present invention, a reinforcing structure of the core means in particular that the core provides reinforcement for the sheath, in particular in that the core has greater stability or strength than the sheath. In particular, the reinforcement may relate to the tensile strength of the filament.

The structure of the filament described above, and thus the structure of the implant, can make it possible to combine a particularly effective therapeutic effect, especially in the field of hyperthermic therapy, with a particularly advantageous applicability of the implant.

This is because the implant can be heated intracorporally by the magnetically heatable filler via electromagnetic excitation. The temperature achieved is in particular proportional to or dependent on the particle loading. Sufficiently high heating to the therapeutically effective temperature of 41° C. to 44° C., for example to destroy tumor tissue, requires a high particle loading of the filler while complying with medical safety limits with regard to the parameter selection of the electromagnetic field. This high loading traditionally poses a major problem for the processability into a monocomponent fiber. This is due to the fact that the tensile strength of the fiber decreases with increasing particle loading, as does the filter life during the manufacture of the fiber or filament.

In the implant described here and thus in the bicomponent fiber manufacturing approach, in addition to the particle-loaded functional component or the particle-loaded functional area in the sheath, there is a second component or a second area by means of which a significant improvement in mechanical strength, in particular tensile strength, can be ensured both during manufacture, in particular during the spinning process during manufacture, and after completion.

This also applies to structures known in the prior art, for example, which do not have a coating at the fiber or filament level, but in which an already formed tubular overall structure of a stent graft is coated.

Thus, it becomes possible to obtain a significantly improved stability compared to the state of the art, which significantly improves the application. In particular, the improved stability can be achieved at high particle loadings, which is necessary for effective use in hyperthermal therapy, especially for tumor control. This is because high particle loading in particular can enable a temperature of the magnetically heatable filler through electromagnetic excitation which can permit particularly reliable destruction of the tumor tissue.

A high degree of flexibility or freedom in particle loading is thus made possible, since the decreasing strength of the sheath at high loadings can be compensated for by the properties of the core and thus the reinforcing structure formed by the core. Furthermore, it becomes possible to allow sufficient elasticity under bending stress. This is because the materials can become brittle when highly loaded with filler, which can limit the bending radius. According to the invention, production can thus be improved, since, for example, in the single-thread braiding process, which can be used, for example, to produce a stent, the achievable angles at the deflection pins can be improved. Thus, the applicability can be improved. This is important because a certain temperature must be attainable in order to be therapeutically effective, but a temperature of 44° C. should not be reached or exceeded in certain applications in order not to cause lasting damage to healthy surrounding tissue. This hyperthermia treatment makes use of the fact that tumor tissue reacts more sensitively to an increased temperature than healthy normal tissue. In order to reach a suitable temperature window under conditions that are particularly gentle for the human body, it is therefore advantageous to have a high particle loading, which can be achieved according to the invention without loss of the mechanical properties of the implant as a whole, as mentioned above, for example.

Thus, for example, an interplay of the thickness of the core or the reinforcement layer in relation to the necessary particle loading can always result in an implant that is as low in material as possible and thus low in space, which can nevertheless permit a high degree of effectiveness for the desired application.

In this way, moreover, a similarly high level of heating can be achieved in comparison with the monocomponent fiber, while at the same time reducing costs, since only part of the fiber cross-section needs to have a particle loading.

Thus, it is clear that the advantages described above can be particularly beneficial in hyperthermic therapy. In this regard, the following should be mentioned. The approach of hyperthermia in that, for example, cancer cells react more sensitive to heat than healthy body cells. Temperature increases to more than 43° C. or 44° C., however, lead to cell death by necrosis even of the healthy body cells.

In this regard, it should be mentioned that cellular necrosis, which can occur at temperatures above 43° C., such as above 44° C., is often the result of very severe damage to a cell, which reacts with cell membrane loss. An inflammatory response occurs, resulting in inflammation and scarring. Apoptosis, approximately in a temperature range of 41° C. to 43° C. or up to 44° C., is a form of programmed cell death with gradual decomposition of the cell. The DNA is fragmented. An inflammatory reaction is avoided and the membrane itself remains intact. In general, it is assumed that the toxic effect of hyperthermia (apoptotic or necrotic) is caused by denaturation of thermolabile proteins in the cytoplasm and intranuclear. There is a co-effect by further aggregation with other proteins or DNA, which impedes cell replication.

Thus, in particular, a defined temperature range that can be precisely set in relation to the temperature window, as is possible according to the invention, is of great advantage.

In addition, hyperthermia treatment provides better blood circulation of the tumor and sensitizes the tissue for the absorption of drugs as well as the rays of a radiation treatment, such as radiotherapy. Decisive for the effect of hyperthermia treatment are, among other things, the level of temperature in the target area as well as the duration of application. To prevent necrosis, the magnetically heated filler is used. Here, cell death occurs through apoptosis: apoptosis is part of the metabolism of every cell and is therefore also called natural, controlled cell death. As explained above, there is no inflammatory reaction (as with necrosis) and it is also ensured that the affected cell dies without damaging the neighboring tissue. Thus, the implant of the invention provides a gentle and effective method for hyperthermic therapy.

In summary, allowed is a measure for precise interval ablation therapy, accurate in location, by the use of a magnetically or inductively heatable implant structure to treat occlusions of hollow organs or vessels caused by tumor growth without the need for multiple surgical interventions.

Preferably, the magnetically heatable filler can be a superparamagnetic filler. The superparamagnetic effect of nanoferrites, for example, describes a magnetic property of very small particles of a ferromagnetic or ferrimagnetic material. If these do not exhibit any permanent magnetization even at temperatures below the Curie temperature after a previously applied magnetic field has been switched off, this is referred to as a superparamagnetic effect. An accumulation of nanoferrites in the polymer matrix therefore behaves macroscopically like a paramagnet' but nevertheless has the high magnetic saturation of a ferromagnet and accordingly reacts like a soft magnetic ferromagnet to inductive fields. In contrast to a paramagnet, it is not individual atoms but small magnetic particles that change their direction of magnetization independently of each other. Such superparamagnetic materials, in particular superparamagnetic nanoferrite particles with an adjustable saturation temperature, are thus preferably used to control local heating.

The advantage of such fillers may be that they enable the structure to be heated to an adjustable saturation temperature in a particularly defined manner and/or within a short period of time. As a result, hyperthermal therapy can be carried out in a particularly gentle manner using an implant according to the invention.

Examples of such superparamagnetic fillers include in particular ferrites, such as superparamagnetic iron oxide particles, for example magnetite or maghemite.

In particular, it may be advantageous if the filler, especially the superparamagnetic filler, has a crystallite size, which may also be referred to as core size or magnetic core size, in a range from greater than or equal to 3 nm to less than or equal to 100 nm, such as greater than or equal to 10 nm to less than or equal to 30 nm, wherein the crystallite size at which a material exhibits superparamagnetic properties may be strongly material-dependent. Such nanoparticles, also referred to as magnetic nanoparticles (MNP), can allow the described advantages in diagnostic (contrast agents in magnetic resonance imaging (MRI)) and therapeutic applications due to their physical properties particularly effectively.

This is because nanoparticles tend to form agglomerates with a size of a few micrometers (macroscopic agglomerates), for example ≤10 μm, due to their magnetic attraction and the large surface-to-volume ratio. In the production of nanocomposites, the formation of agglomerates in the production process can only be influenced to a limited extent or at great expense. The agglomerates act like imperfections and significantly affect the properties of the resulting nanocomposites. One possibility for improving the material properties lies in a homogeneous distribution of the particles in the end product. Two manufacturing processes are currently used industrially to produce nanocomposites, the melt-mixing process by means of extrusion, for example as a melt-spinning process with a twin-screw extruder, and the solution-mixing process. In-situ polymerization, particle functionalization or ultrasonic waves are used to homogenize the nanoparticles in the matrix.

The spinning process is particularly advantageous, in which a second component, the core, is spun out in addition to the particle-loaded functional component, the sheath, for the manufacture of the implant according to the invention, through which a significant improvement in mechanical strength can be ensured both during the spinning process and after completion. Thus, in particular the coextrusion of two materials is used in the melt spinning process.

The spinning process follows the melt mixing process with the twin screw extruder. This can be a one-step process, but also a two-step process. The product of the melt mixing process, also called compounding, is pellets. This is then spun out into fibers in the melt spinning process.

It may be further preferred that the implant has a tubular structure, i.e. in particular a duct-like or hose-like structure. For example, the implant can be a stent. In this case, the filament built up from a core-sheath structure can thus be processed by fiber processing processes to form the tubular structure. In particular, in this embodiment, the implant may be advantageous for hyperthermal therapeutic applications. Particularly preferably, this structure may be effective in insertion into hollow organs or into vessels, such as in cancer therapy. The implant described herein thus enables particularly advantageous properties, especially in the therapy of cancer patients or also for hyperthermally combating stenoses.

In this regard, it should be noted that cancer is the second highest cause of death in Germany. The tumor mass often infiltrates or constricts vessels and hollow organs, such as veins, the trachea, bile ducts, ureters and urethra. Stenoses are often caused by intimal hyperplasia, the proliferation of cells. This, in addition to stent thrombosis, is a typical complication after stent implantation. Pre-described can lead to a life-threatening situation. If possible, the tumor mass is removed surgically, and stenoses occurring in the cardiovascular area are often treated with drugs. However, local recurrences often lead to a new occlusion or restenosis. In this regard, metal stents are often used to keep the hollow body or vessel open, but this is often only temporarily effective, thus necessitating renewed surgery.

The implant described here is based on the fact that the tumor or tumor tissue can be destroyed by local hyperthermia, or that any tumor-related or stenosis-related constrictions or occlusions of the hollow organ or vessel can be removed, thereby exposing the hollow structures again.

This is made possible in a particularly advantageous manner by an implant according to the present invention. This is because, on the one hand, its increased stability can allow it to be inserted into the hollow organ or a vessel without any problems. In addition, the hyperthermal properties can enable defined heating of the implant, which has an effective effect on gentle therapy.

In addition, the filament can form an open-pored implant that can maintain the basic advantages, namely the retention of vessel diameter, while circumventing the disadvantages, namely in particular the ingrowth of tumor tissue, by hyperthermally removing ingrown tissue.

In particular, when the implant is inserted in a hollow organ or a vessel, it can be introduced to the affected site via a catheter system. For example, self-expansion can be used to expand the implant in the lumen and fix it in the vessel or organ wall. After insertion, the implant is locally heated via an alternating magnetic field, several times if necessary, as already explained above. The treatment of a tumor growing into a hollow organ, vessel or other cavity can thus be carried out non-invasively via electromagnetic heating, as already described. Thus, the patient could be spared the sometimes dangerous repeated surgical interventions. Since regular revision operations would no longer be necessary, there would also be a significant cost saving for the healthcare system.

Further advantageously, the filament can have a crossed or entangled structure. Thus, the filament can be processed into the implant, in particular by fiber processing processes known per se, and in particular not be a nonwoven. In this way, a defined and equally stable structure can be produced, which can improve its use as an implant.

It may be particularly preferred that the filament has a braided structure. In particular, a braided structure may have advantages for therapy in a hollow organ or in a vessel. This is because, in particular, a textile stent can be made possible in a braided structure, which has a high flexibility and undergoes a low mechanical stress on the fibers during manufacture. Furthermore, a braid in particular can be easily compressed to be inserted into a hollow organ or a vessel, and can thereby maintain its desired non-compressed shape in the hollow organ due to a sufficient mechanical restoring force. As a result, a braid can have advantages over other products made by fiber-processing methods or even over nonwovens or nonwoven fabrics.

It may further be preferred if the magnetically heatable filler is present in the sheath in a proportion of greater than or equal to 0.1% by weight to less than or equal to 90% by weight, preferably greater than or equal to 3% by weight to less than or equal to 30% by weight. In particular, in this embodiment, the filler can be heated such that the implant is heated to the desired temperature described above in a range from about 41° C. to 44° C. In this regard, the implant according to the invention, particularly in this embodiment, may have reduced mechanical properties, such as reduced stability, in a monocomponent structure, i.e., without the reinforcing layer, so that the present invention may have effective advantages, particularly in this embodiment.

With regard to the polymeric matrix material, it can be advantageous that this is selected from the list consisting of polypropylene, polyethylene terephthalate, polyvinylidene fluoride, polyethylene, polyamide and thermoplastic polyurethane. It has been shown that these polymers in particular are suitable for homogeneously dispersing a filler. Furthermore, they can be sufficiently heated by the filler, so that they have no or only limited negative influence on hyperthermic therapy. In addition, the polymers described here are also well suited as implants due to their inertness and are not or not significantly degraded by the human body, so that long-term use as implants is also possible.

Additionally or alternatively, it can be provided that the core forms a polymeric reinforcement structure comprising the same polymeric matrix material, for example consisting of this, as the sheath. In this embodiment, the compatibility as an implant can be further increased, since only a material that comes into contact with the body needs to be introduced into the body, and any repulsive reactions or the risk thereof can thus be further avoided.

In addition, a particularly high level of stability can be achieved, especially in this embodiment, since the same materials can adhere to each other or be bonded to each other particularly well in the core-shell structure used. Finally, production processes can be simplified, which can save costs.

With regard to the stability to be achieved, it can also be advantageous that the core is free of the magnetically heatable filler. In this embodiment, the reinforcement structure can have a particularly high stability or particularly advantageous mechanical properties, since the stability is not reduced at all by fillers present in the polymeric material. As a result, the thickness of the filaments can be reduced or, with the same thickness, an increasing loading of the sheath with a filler can be achieved.

Further, it may be preferred that the ratio of the thickness of the core to the thickness of the sheath lies in a range of greater than or equal to 1/19 to less than or equal to 19/1. For example, the sheath may have a proportion, based on the combination of core and sheath, of greater than or equal to 30% by weight to less than or equal to 70% by weight. In principle, when the ratio is in the present range, it is possible to provide a reinforcement sufficient to allow easy application as an implant, such as in hollow organs or vessels, while still allowing a high loading to achieve a therapeutically effective temperature.

The same can be achieved if the core-sheath structure, or the implant, respectively, forms a two-layer structure, i.e. the filament consists of the sheath and the core. In this embodiment, too, additional material can be waived.

With regard to further technical features and advantages of the implant, reference is made to the description of the arrangement, to the figures and to the description of the figures, and vice versa.

The invention further relates to an arrangement comprising a radiation source for emitting electromagnetic radiation and an implant, wherein the implant comprises a magnetically heatable filler. The arrangement is characterized in that the implant is configured as previously described.

Such an arrangement allows, that through the radiation source, the magnetically heatable filler is inductively heated in a defined and reproducible manner by magnetic relaxation processes, and the implant can thus be used for hyperthermal therapy in a defined manner.

For this purpose, it can be advantageous, especially when used in the body, that the implant and the radiation source are matched to each other in such a way that the implant can be heated by electromagnetic radiation emitted by the radiation source, at least in the sheath, to a temperature that lies in a range from 40° C. to 100° C., for example from 41° C. to 44° C. This can enable, for example, effective and equally gentle destruction of cancerous tissue.

Appropriate tuning of emitted radiation to the implant can be made possible on the electromagnetic radiation side in particular by setting a suitable frequency. Exemplary frequencies and field amplitudes cover a range from 10 kHz to 1 MHz and 1 kA/m to 100 kA/m.

Such ranges can be advantageous because, with a suitable combination of frequency and field amplitude, the unintentional heating of tissue can be counteracted particularly effectively by the formation of so-called eddy currents. In particular, it is taken into account that the energy deposition of the tissue is frequency-dependent.

Appropriate tuning of emitted radiation, i.e. the frequency and field amplitude as well as the direction of the magnetic field, to the implant can be done on the part of the implant in particular by shaping the particle properties, e.g. their size, crystallinity, magnetic behavior, in particular their magnetic relaxation, their stabilizing shell, which affects the homogeneous distribution of small or no agglomerates in the polymer. The design of the particles also provides for an arrangement of individual particles in the polymer in the form of a chain or as an agglomerate, which results in the enhanced response to the applied magnetic field.

With regard to further technical features and advantages of the arrangement, reference is made to the description of the implant, to the figures and to the description of the figures, and vice versa.

The following is an exemplary explanation of the invention with reference to the accompanying drawings, wherein the features shown below may each individually or in combination constitute an aspect of the invention, and wherein the invention is not limited to the following drawing, description and embodiment.

It show:

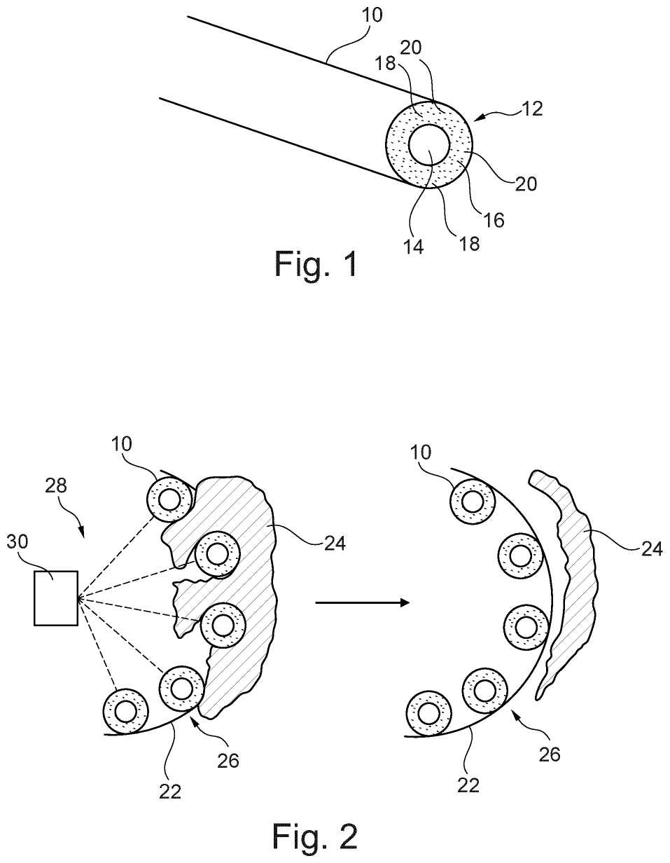

FIG. 1a schematic view of a filament for an implant according to the present invention; and

FIG. 2 the mode of action of an implant according to the invention.

FIG. 1 shows a schematic view of a filament 10 for an implant 26 according to the present invention.

In particular, the implant 26 serves for implanting into a body, particularly into a hollow organ 22 of a body, as shown in FIG. 2, to fight against hollow organ tumors. In addition, the implant 26 may also be inserted into a vessel.

The implant 26 is constructed from the filament 10, for example in a braided structure. The filament 10 has at least one polymeric matrix material 18 in which a magnetically heatable, in particular superparamagnetic, filler 20 is arranged.

Further, FIG. 1 shows that the filament 10 has a cross-section with a core-sheath structure 12 such that the core 14 forms a polymeric reinforcing structure.

It is shown that the core 14 is arranged filamentary and as a fiber or filament, and the sheath 16 has a hose-like structure and at least partially envelops the core 14.

The sheath 16 comprises the polymeric matrix material 18 in which the magnetically heatable, in particular nanoscale, filler 20 is arranged. It can be seen that the loading of the filler 20 in the sheath 16 is greater than in the core 14. In particular, the core 14 is free of the filler 20. Furthermore, the core-sheath structure 12 or the filament 10 is designed in particular as a two-layer structure.

In particular, the magnetically heatable filler 20 may be present in the sheath 16 in an amount from greater than or equal to 0.1 wt % to less than or equal to 90 wt %. Further, the matrix material 18 may be selected from the group consisting of polypropylene, polyethylene terephthalate, polyvinylidene fluoride, polyethylene, polyamide, and thermoplastic polyurethane. The material of the core 14 may be formed from the same aforementioned material.

As indicated in FIG. 2, the filament 10, which can also be referred to as a bicomponent fiber, can be processed into an implant 26, in particular a stent, with the aid of textile manufacturing processes. Preferably, braiding is mentioned here as a manufacturing process. The implant 26 can then be used, as shown in FIG. 2, in infiltrated tumors in vessels and hollow organs 22 to keep the vessels and hollow organs 22 open and for hyperthermic treatment of the tumors by destroying tumor tissue 24.

To implement this form of therapy, the implant 26 is designed as a textile stent that can be heated by magnetic induction. Here, the filament braided structure is used as the textile stent. This braided structure consists of polymer fibers having incorporated nanoferrites as filler 22. The nanoferrites to be used are synthesized and compounded together with the polymer on a twin screw extruder to form a spinnable masterbatch. This masterbatch is then spun into inductively heatable fibers using the melt spinning process. In particular, a coextrusion of core material and sheath material is carried out to create the core-sheath structure. The implant 26 or the stent, respectively, is advanced via a catheter system to the corresponding location in the body or in the hollow organ 22 or vessel and then expanded by means of self-expansion.

When excited in an electromagnetic field, the nanoferrites convert the absorbed energy of the field into heat and release it to the environment. This is made possible, for example, by using a radiation source 30 which emits electromagnetic radiation in such a way that the filler 20 heats up to preferably 43° C. The radiation source 30 can form a coherent or coordinated arrangement 28 with the implant 26.

The resulting local hyperthermia can destroy ingrown tumors or corresponding tumor tissue 24 around the implant 26. As described, this is possible in particular by using specific superparamagnetic nanoferrites with an adjustable saturation temperature as filler 20. The achievable surface temperature depends largely on the parameters of the magnetic field, which must be adjusted to the properties of the nanoferrites and the fibers incorporated with nanoferrites, and on the level of particle loading or filler loading of the filament 10. The parameter selection of the electromagnetic field is limited by compliance with medical safety limits. However, these are readily achievable according to the present invention, since the filler loading can be selected to be sufficiently high due to the reinforcing layer of the core 14.

The nanoferrites release the absorbed inductive energy as heat via the polymer fibers to the tumor tissue 24, acting as an intrinsic thermostat. In this way, the tumor tissue 24 is destroyed by a local increase in temperature, as shown in FIG. 2.

### REFERENCE SIGNS

10 filament
12 core-sheath structure
14 core
16 sheath
18 matrix material
20 filler
22 hollow organ
24 tumor tissue
26 implant
28 arrangement
30 radiation source

1. Implant for implanting in a body, in particular in a hollow organ or a vessel of a body, the implant being composed of a filament which comprises at least one polymeric matrix material in which a magnetically heatable filler is arranged, the filament having a cross section with a core-sheath structure characterized in that the core forms a polymeric reinforcing structure, and in that the sheath comprises the polymeric matrix material in which the magnetically heatable filler is disposed, the loading of the filler being greater in the sheath than in the core.

2. Implant according to claim 1, characterized in that the core is thread-like and the sheath has a hose-like structure and at least partially envelops the core.

3. Implant according to claim 1, characterized in that the magnetically heatable filler is a superparamagnetic filler.

4. Implant according to claim 1, characterized in that the filler has a crystallite size in a range from greater than or equal to 3 nm to less than or equal to 100 nm.

5. Implant according to claim 1, characterized in that the filament has a crossed structure or an entangled structure or that the filament has a braided structure.

6. Implant according to claim 5, characterized in that the implant forms a tubular structure.

7. Implant according to claim 1, characterized in that the magnetically heatable filler is present in the sheath in a proportion of greater than or equal to 0.1% by weight to less than or equal to 90% by weight.

8. Implant according to claim 1, characterized in that the polymeric matrix material is selected from the group consisting of polypropylene, polyethylene terephthalate, polyvinylidene fluoride, polyethylene, polyamide and thermoplastic polyurethane.

9. Implant according to claim 1, characterized in that the core forms a polymeric reinforcing structure comprising the same polymeric matrix material as the sheath.

10. Implant according to claim 1, characterized in that the core is free of the magnetically heatable filler (20).

11. Implant according to claim 1, characterized in that the core-sheath structure is produced by a coextrusion process.

12. Implant according to claim 1, characterized in that the core-sheath structure forms a two-layer structure.

13. Arrangement of a radiation source for emitting electromagnetic radiation and an implant, wherein the implant comprises a magnetically heatable filler, characterized in that the implant is configured according to claim 1.

14. Arrangement according to claim 13, characterized in that the implant and the radiation source are matched to each other in such a way that the implant is heatable by electromagnetic radiation emitted by the radiation source, at least in the sheath, to a temperature which lies in a range from 40° C. to 100° C.

15. Arrangement according to claim 13, characterized in that the radiation source is arranged to emit radiation of a frequency in a range from 10 kHz to 1 MHz at a field amplitude range from 1 kA/m to 100 kA/m.
